(19) United States
(12) Patent Application Publication
(10) Pub. No.: US 2009/0306107 A1
(43) Pub. Date: Dec. 10, 2009

(54) Organic Compounds

(75) Inventors: Lawrence Blas Perez, Hopkinton, MA (US); Tao Sheng, Westwood, MA (US)

Correspondence Address: CARSTENS & CAHOON, LLP, P O BOX 802334, DALLAS, TX, 75380, US

(73) Assignee: NOVARTIS AG, Basel (CH)

(21) Appl. No.: 11/908,913

(22) PCT Filed: Mar. 17, 2006
(86) PCT No.: PCT/US2006/009713

§ 371 Date: Sep. 17, 2007

Related U.S. Application Data
(60) Provisional application No. 60/662,889, filed on Mar. 17, 2005.

(51) Int. Cl. A61K 31/519; C07D 471/04; A61P 35/00

(52) U.S. Cl. 514/267; 544/250

(57) ABSTRACT

The present invention relates to compounds that are useful to inhibit, regulate and/or modulate tyrosine and serine/threonine kinase and kinase-like proteins, such as RAF kinase, a serine/threonine kinase that functions in the MAP kinase signaling pathway. The application is also concerned with compositions which contain these compounds, and methods of using them to treat tyrosine and serine/threonine kinase and kinase-like dependent diseases, such as angiogenesis, cancer and cardiac hypertrophy, and with other subject matter.

This application is the National Stage of Application No. PCT/US2006/009713 filed on Mar. 17, 2006 based on provisional 60/662,889 filed on Mar. 17, 2005. The contents are incorporated herein by reference in their entirety.

### SUMMARY

The present invention relates to compounds that are useful to inhibit, regulate and/or modulate tyrosine and serine/threonine kinase and kinase-like proteins, such as RAF kinase, a serine/threonine kinase that functions in the MAP kinase signaling pathway. The application is also concerned with compositions which contain these compounds, and methods of using them to treat tyrosine and serine/threonine kinase and kinase-like dependent diseases, such as angiogenesis, cancer and cardiac hypertrophy, and with other subject matter.

### BACKGROUND

Cells communicate various aspects of their extracellular environment to the nucleus by using various signal transduction pathways. Many of these signals are transmitted by protein kinases which activate various factors through the transfer of phosphate groups. Disruption of signal transduction by inhibiting appropriate kinase activity can have a clinical benefit as has been demonstrated by imatinib, an inhibitor of bcr-abl kinase, which is marketed as its mesylate salt under the brand GLEEVEC (in the United States) or GLIVEC.

The MAP kinase signaling pathway is known in the art as one of the pathways for growth factors to send their signal to proliferate from the extracellular environment to the cell nucleus. The growth factors activate transmembrane receptors located on the cell surface which in turn start a cascade whereby RAS is activated and recruits RAF kinase to the membrane where it is activated and in turn activates MEK kinase which then activates ERK kinase. Activated ERK kinase can move to the nucleus where it activates various gene transcription factors. Aberrations in this pathway can lead to altered gene transcription, cellular growth and contribute to tumorigenicity by negatively regulating apoptosis and transmitting proliferative and angiogenic signals. Inhibitors of RAF kinase have been shown to block signaling through the MAP kinase signaling pathway.

The RAF kinase family is known to have three members designated C-RAF, also known as RAF-1, B-RAF and A-RAF. It has been reported that B-RAF kinase is commonly activated by one of several somatic point mutations in human cancer, including 59% of the melanoma cell lines tested. See, Davies, H. et al, Nature 417, 949-954 (2002). This invention relates to the discovery of a class of compounds that efficiently inhibit one or more members of the RAF kinase family.

The RAF kinase inhibiting property of the compounds makes them useful as therapeutic agents for the treatment for proliferative diseases characterized by an aberrant MAP kinase signaling pathway, particularly many cancers characterized by overexpression of RAF kinase or an activating mutation of RAF kinase, such as melanoma having mutated B-RAF, especially wherein the mutated B-RAF is the V599E mutant. The present invention also provides a method of treating other conditions characterized by an aberrant MAP kinase signaling pathway, either with wild type B-RAF or mutant B-RAF, and particularly where B-RAF is mutated, for example benign Nevi moles having mutated B-RAF, with the compounds.

### DESCRIPTION

The present invention relates in one aspect to compounds of formula (I) and to a method of treating a patient having a disease characterized by excessive signaling through tyrosine and serine/threonine kinase and kinase-like proteins, which comprises administering to the patient an effective kinase inhibiting amount of a compound of formula (I). A preferred target in a signaling pathway is B-RAF, especially mutant B-RAF.

One aspect of the invention relates to compounds which are described by formula (I) or pharmaceutically acceptable salts, esters, prodrugs or N-oxides thereof.

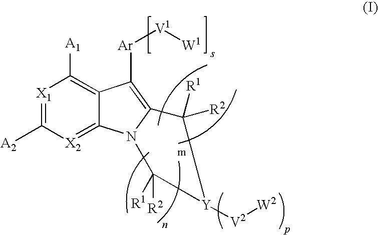

wherein
A₁ and A₂ are each independently selected from H, NR^{a}R^{b}, OR^{c}, SR^{c} or alkyl, e.g., lower alkyl or aryl;
where R^{a} and R^{b} are each independently selected from hydrogen; OH; hydrocarbyl (for example alkyl e.g. lower alkyl, alkenyl e.g. lower alkenyl, aryl or cycloalkyl) or hydrocarbyloxy (for example alkoxy e.g. lower alkoxy, or aryloxy), the hydrocarbyl moieties optionally being substituted by one or more substituents selected from halo and hydroxy (as for example in the case of CF₃); mercapto; guanidine; NH₂; NHR^{d}; N(R^{d})₂,
where R^{d} is hydroxy or alkyl, e.g. C₁ to C₄alkyl;
where R^{c} is selected from hydrogen and hydrocarbyl (for example alkyl e.g. lower alkyl, alkenyl e.g. lower alkenyl, aryl or cycloalkyl), the hydrocarbyl optionally being substituted by one or more substituents selected from halo and hydroxy (as for example in the case of CF₃);
X₁ and X₂ are each independently selected from N or CR^{c} (e.g. CH);
m, n and s are each independently selected from 0, 1, 2, 3, 4, 5; and
p is 0 or 1, such that p+s≧1 (preferably, p=0 and s=1);
Y is selected from O, S, N or C and, where Y is O or S, p=0;
V¹ and V² are each independently a linking moiety selected from one or more of lower alkyl, amine, ether, amide, ester, urea, carbamate, sulphonamide or a direct bond;
W¹ and W² are each independently selected from H, alkyl, or a substituted or unsubstituted cyclic group, e.g. aryl group;
Ar is a substituted or unsubstituted cyclic group, e.g. aryl group; and
each R¹ and R², if present, are independently selected from hydrogen, lower alkyl, lower alkoxy, halo, hydroxyl, amino and mono- or di-lower alkylamino, wherein when n or m is ≧1, each R¹ and R² may be the same or different.

Exemplary compounds of the invention include the following
N-[3-(1-Amino-5,6,7,8-tetrahydro-2,4,4b-triazafluoren-9-yl)-phenyl]benzamides;
N-[3-(1-amino-5,6,7,8-tetrahydro-2,4,4b-triazafluoren-9-yl)-phenyl]-3-(1,1,2,2-tetrafluoro-ethoxy)benzamide;
N-[3-(4-Amino-7-but-3-enyl-7H-pyrrolo[2,3-D]pyrimidin-5-yl)-phenyl]-benzamide;
N-[3-(4-Amino-6-bromo-7-but-3-enyl-7H-pyrrolo[2,3-D]pyrimidin-5-yl)-phenyl]-benzamide;
N-[3-(1-Amino-5,6,7,8-tetrahydro-2,4,4b-triazafluoren-9-yl)-phenyl]benzamide;
9-(3-Amino-phenyl)-5,6,7,8-tetrahydro-2,4,4b-triazafluoren-1-ylamine;
1-[3-(4-Amino-7-cyclopentyl-7H-pyrrolo[2,3-d]pyrimidin-5-yl)-phenyl]-3-(3-trifluoromethyl-benzoyl)-urea;
N-[3-(1-Amino-5,6-dihydro-8H-7-oxa-2,4,4b-triaza-fluoren-9-yl)-phenyl]-3-morpholin-4-yl-5-trifluoromethyl-benzamide; and
N-[3-(1-Amino-5,6,7,8-tetrahydro-2,4,4b-triazafluoren-9-yl)-phenyl]-3-(1,1,2,2-tetrafluoro-ethoxy)benzamide.

Within the context of the present disclosure, the general terms used herein to describe compounds of the present invention have the following meanings, unless indicated otherwise.

Alkyl preferably has up to 20, more preferably up to 12 carbon atoms and is linear or branched one or more times; preferred is lower alkyl, especially C₁, C₂, C₃, or C₄ alkyl, in particular methyl, ethyl or i-propyl or t-butyl, where alkyl may be substituted by one or more substituents. Unsubstituted alkyl, preferably lower alkyl, is especially preferred.

The term “lower” when referring to the alkyl portion of lower alkyl, lower alkoxy, mono- or di-lower alkyl amino (NHR^{d}, N(R^{d})₂), lower alkyl thio (SR^{c}) and other substituents with an alkyl portion denotes a radical having up to and including a maximum of 7, especially 1, 2, 3 or 4 carbon atoms, the radicals in question being unbranched or branched one or more times, for example n-butyl, sec-butyl, tert-butyl, n-propyl, isopropyl, methyl or ethyl. Such alkyl substituents are unsubstituted or substituted by halogen, hydroxy, nitro, cyano, lower alkoxy, C₃, C₄, C₅, C₆ or C₇ cycloalkyl, amino, or mono- or di-lower alkyl amino, unless otherwise indicated.

Halo-lower alkyl, halo-lower alkyloxy, halo-lower alkylthio and the like refer to substituents having an alkyl portion wherein the alkyl portion is mono- to completely substituted by halogen. Halo-lower alkyl, halo-lower alkyloxy, halo-lower alkylthio and the like are included within substituted lower alkyl, substituted lower alkoxy, substituted lower alkylthio and the like.

Alkyl may be optionally interrupted by one or more in-chain heteroatoms, for example —O—, thus forming, for example, an ether linkage.

Cyclohydrocarbyl includes cycloalkyl and cycloalkenyl.

Cycloalkyl is preferably C₃-C₁₀-cycloalkyl, especially cyclopropyl, dimethylcyclopropyl, cyclobutyl, cyclopentyl, cyclohexyl or cycloheptyl, cycloalkyl being unsubstituted, or substituted by one or more, especially 1, 2 or 3, substituents.

Heterocycloalkyl is the same as cycloalkyl except that at least one of the in-ring carbon atoms is replaced with a heteroatom selected from N, O or S. The heteroatom may be N.

Cycloalkenyl and heterocycloalkenyl are the same as cycloalkyl and heterocycloalkyl respectively, except that the have at least one in-ring double bond, i.e. at least one degree of unsaturation.

Substituents of, for example, alkyl or cycloalkyl, may be selected from one or more, especially up to three, substituents primarily from the group selected from halogen, especially fluorine, amino, N-lower alkylamino, N,N-di-lower alkylamino, N-lower alkanoylamino, hydroxy, cyano, carboxy, lower alkoxycarbonyl, and phenyl-lower alkoxycarbonyl. Trifluoromethyl is especially preferred.

Among the moieties corresponding to substituted alkyl, hydroxy-lower alkyl, especially 2-hydroxyethyl, and/or halo-lower alkyl, especially trifluoromethyl or 2,2,2-trifluoroethyl, are especially preferred.

An aryl group is an aromatic radical and may be heterocyclic or carbocyclic. Preferably, aryl is carbocyclic. Preferably aryl has a ring system of not more than 16 carbon atoms and is preferably mono-bi- or tri-cyclic and may be fully or partially substituted. A substituted carbocyclic aryl group is generally an aryl group that is substituted with from 1-5, preferably 1 or 2, substituents. Preferably, aryl is selected from phenyl, naphthyl, indenyl, azulenyl and anthryl, and is preferably in each case unsubstituted or substituted by, for example lower alkyl, especially methyl, ethyl or n-propyl, halo (especially fluoro, chloro, bromo or iodo), substituted lower alkyl, for example halo-lower alkyl (especially trifluoromethyl), hydroxy, lower alkoxy (especially methoxy), substituted lower alkoxy, for example halo-lower alkoxy (especially 2,2,2-trifluoroethoxy) or amino-lower alkoxy (especially 2-amino-ethoxy), lower alkanoyl, carbamoyl, N-mono- or N,N-di-lower alkyl substituted carbamoyl, wherein the lower alkyl substituents may be unsubstituted or further substituted, for example lower alkyl (especially methyl or ethyl) carbamoyl or N-(hydroxy-lower alkyl)-carbamoyl (especially N-(2-hydroxyethyl)-carbamoyl), sulfamoyl-substituted aryl, especially a corresponding substituted or unsubstituted phenyl, amino, mono- or di-lower alkyl substituted amino, wherein the lower alkyl substituents may be unsubstituted or further substituted by those substituents listed above for alkyl groups, nitro, cyano, mercapto, lower alkylthio, halo-lower alkylthio, heterocyclyl, heteroaryl, heterocyclylalkyl or heteroarylalkyl. An aryl carbocyclic group especially comprises 3, 4, 5, 6 or 7 in ring carbon atoms.

Heterocyclyl (or heterocyclic group) is preferably a heterocyclic radical that is unsaturated, saturated or partially saturated and is preferably a monocyclic or, in a broader aspect of the invention, a bicyclic or tricyclic ring; has 3 to 24, more preferably 4 to 16 ring atoms. A heterocycle is especially a 5 to 7 membered aromatic ring comprising from 1 to 3 heteroatoms selected from N, O and S.

Heteroaryl-lower-alkylene and heterocyclic-lower-alkylene are substituents of the formula het-C₁-C₄-alkylene- where het is a heteroaryl or heterocyclic radical.

Aryl, where containing a heteroatom, is heterocyclic. Heterocyclic radicals are especially selected from the group consisting of oxiranyl, azirinyl, 1,2-oxathiolanyl, imidazolyl, thienyl, furyl, tetrahydrofuryl, pyranyl, thiopyranyl, thianthrenyl, isobenzofuranyl, benzofuranyl, chromenyl, 2H-pyrrolyl, pyrrolyl, pyrrolinyl, pyrrolidinyl, imidazolyl, imidazolidinyl, benzimidazolyl, pyrazolyl, pyrazinyl, pyrazolidinyl, pyranyol, thiazolyl, isothiazolyl, dithiazolyl, oxazolyl, isoxazolyl, pyridyl, pyrazinyl, pyrimidinyl, piperidyl, piperazinyl, pyridazinyl, morpholinyl, thiomorpholinyl, indolizinyl, isoindolyl, 3H-indolyl, indolyl, benzimidazolyl, cumaryl, indazolyl, triazolyl, tetrazolyl, purinyl, 4H-quinolizinyl, isoquinolyl, quinolyl, tetrahydroquinolyl, tetrahydroisoquinolyl, decahydroquinolyl, octahydroisoquinolyl, benzofuranyl, dibenzofuranyl, benzothiophenyl, dibenzothiophenyl, phthalazinyl, naphthyridinyl, quinoxaloyl, quinazolinyl, quinazolinyl, cinnolinyl, pteridinyl, carbazolyl, α-carbolinyl, phenanthridinyl, acridinyl, perimidinyl, phenanthrolinyl, furazanyl, phenazinyl, phenothiazinyl, phenoxazinyl, chromenyl, isochromanyl and chromanyl, each of these radicals being unsubstituted or substituted by one to two radicals. Heterocycle, is especially imidazole, pyrrole, oxazole, isoxazole, pyridine.

Important substituents on heterocycle are those selected from the group consisting of halogen, for example, fluorine or chlorine; mono- or di-lower alkyl-substituted amino wherein the alkyl groups are unsubstituted or substituted by halogen, hydroxy, nitro, cyano, lower alkoxy, C₃-C₇ cycloalkyl, lower alkyl, such as methyl or ethyl; halo-lower alkyl, such as trifluoromethyl; lower alkoxy, such as methoxy or ethoxy; halo-lower alkoxy, for example, trifluoromethoxy and 1,1,2,2-tetrafluoroethoxy; lower alkylthio, such as methylmercapto, halo-lower alkylthio, such as trifluoromethylthio, a heteroaryl radical, heteroaryl-lower-alkylene, a heterocyclic radical or heterocyclic-lower-alkylene.

Halogen is especially fluorine, chlorine, bromine or iodine, more especially fluorine, chlorine or bromine, in particular fluorine.

Hydrocarbyl may have for example up to 20 carbon atoms, preferably up to 12 carbon atoms. Hydrocarbyl groups may be aliphatic, e.g. alkyl, alkenyl or alkynyl; they may be alicyclic, e.g. cycloalkyl; they may be aromatic, e.g. phenyl. Hydrocarbyl groups may contain a combination of two or more moieties selected from aliphatic, alicyclic and aromatic moieties, e.g. a combination of at least one alkyl group and an aromatic group. Aliphatic moieties often contain 1, 2, 3, 4, 5, 6 or 7 carbon atoms, e.g. 1-4. Cyclic moieties often consist of one 5- or 6-membered ring or two 5- or 6-membered rings fused together. In some instances, hydrocarbyl groups may be optionally interrupted by one or more in-chain heteroatoms, for example —O—, thus forming, for example, an ether linkage.

As used herein, the term mercapto defines moieties of the general structure—S—Rₑ wherein Rₑ is H, alkyl, aryl, cyclohydrocarbyl or heterocyclyl as described herein.

As used herein, the term guanidino defines moieties of the general structure —C(NH)NH₂ and derivatives thereof, in particular, where hydrogen is replaced by alkyl, e.g. methyl or ethyl.

Preferably A₁ and A₂ are each independently selected from H, NR^{a}R^{b}, OR^{c}, SR^{c} or lower alkyl. In a further preferred embodiment of the invention, A₁ and A₂ are independently selected from H and NR^{a}R^{b}. Exemplary are NH₂ and NHR^{a} as well as NR^{a}R^{b} groups in which neither R^{a} nor R^{b} is H, e.g. in which both R^{a} and R^{b} are lower alkyl.

More preferably, at least one of A₁ and A₂ is NR^{a}R^{b}, e.g. is NH₂, NHR^{a} in which R^{a} is not H (e.g. is alkyl), or an NR^{a}R^{b} group in which neither R^{a} nor R^{b} is H, e.g. in which both R^{a} and R^{b} are alkyl. In one class of compounds, one of A₁ and A₂ is H and the other is not H; in a sub-class A₁ is not H and A₂ is H. Most preferably, one of A₁ and A₂ is NH₂, and the other of A₁ and A₂ is H.

In a class of compounds, at least one of X₁ and X₂ is N. Preferably each of X₁ and X₂ is N.

In many instances m+n=2 or 3. Most preferably m+n=3.

S is preferably 1.

Y is most preferably C.

In a preferred group of compounds, V² is a direct bond and W² is H.

Preferably V¹ is an amide linker. Included are compounds in which the amide is N-substituted, in particular by a C₁-C₆ hydrocarbyl, e.g. alkyl, group such as methyl, for example.

Where there are plural W¹ groups, they may be the same or different. In one class of compounds, the or each W¹ is a substituted aryl group; there may be 1, 2, 3, 4 or 5 substituents, e.g. 1 or 2 and often just a single substituent. In another class of compounds, the or each W¹ is an unsubstituted aryl group. The aryl group often contains 6 ring-forming atoms and in particular may be phenyl.

As substituents for W¹ may be mentioned groups of the formula—J-R^{f} where J is selected from O, NR^{a}, S, hydrocarbyl (e.g. lower alkyl) or a covalent bond; R^{f} is selected from halo, H, NR^{a}R^{b}, OR^{c}, SR^{c}, where R^{a}, R^{b} and R^{c} are as hereinbefore described and independent of each other. Exemplary substituents include lower alkyl and lower alkoxy, in either case optionally substituted one or more times by halogen, particularly F. Included are compounds in which W¹ is a 3-substituted phenyl group.

In embodiments, there is at least one W¹ group which is substituted by at least one halogen-containing group, particularly fluorine-containing group(s), typically fluoroalkoxy group(s), preferably fluoro lower alkoxy groups, such as fluoromethoxy or fluoroethoxy group(s), for example difluoromethoxy or tetrafluoroethoxy group(s).

In a particular class of compounds, V¹ is an amide linker, V² is a direct bond, W¹ is a substituted phenyl group and W² is H. In another particular class of compounds, V¹ is an amide linker, V² is a direct bond, W¹ is an unsubstituted phenyl group and W² is H

Where V¹ contains more than one linker, it is preferred that one of the linkers is an alkyl group.

Ar is suitably an unsubstituted aryl group, that is an aryl group unsubstituted except for any attached V¹—W¹ moieties.

Ar may be a heterocyclic structure; the heterocycle may be heteroaromatic. The heterocyclic structure may be monocyclic, e.g. having 5 or 6 ring members or it may be a fused heterocycle, for example having two fused rings selected from 5- and 6-membered rings. Exemplary heterocycles are imidazole, pyrrole, oxazole, isoxazole and pyridine.

Alternatively Ar is a carbocyclic group, which may be monocyclic or fused, e.g. it may be a 5- or 6-membered monocycle or a bicyclic structure having two fused rings selected from 5- and 6-membered rings. Ar may be aromatic. A preferred Ar moiety is phenyl.

Where Ar is substituted, that is where Ar has one or more substituents in addition to any V¹—W¹ groups, the further substituent(s) may be selected from halo; OH; hydrocarbyl (for example, alkyl e.g. lower alkyl, alkenyl e.g. lower alkenyl, aryl or cycloalkyl) or hydrocarbyloxy (for example alkoxy e.g. lower alkoxy, or aryloxy), the hydrocarbyl moieties optionally being substituted by one or more substituents selected from halo and hydroxy (as for example in the case of CF₃); mercapto; guanidine; NH₂; NHR^{d}; N(R^{d})₂, where R^{d} is hydrogen, hydroxy or alkyl, e.g. C₁ to C₄ alkyl. An exemplary substituent is fluorine. There may for example be 0, 1 or 2 substituents in addition to any V¹—W¹ groups. Typically there are no further substituents.

Commonly, s is at least 1, e.g. is 0, 1 or 2. Most particularly, s is 1. Typically, Ar is substituted by at the 3-position by a V¹—W¹ group. Preferably, therefore, Ar is substituted—typically 3-substituted—with a single V¹—W¹ moiety, and this preferably forms a substituted amino group, more particularly an amido group, and especially an arylamido group. As described above, the amido group may be N-substituted. An exemplary substituent is a benzamido group. In other words, it is preferred that V¹ is an amido group and W¹ is a phenyl group; as described above, the W¹ phenyl group may be substituted or unsubstituted. It follows that a preferred Ar—(V¹—W¹)ₛ moiety is a 3-(benzamido)phenyl group, whose benzamido part may be substituted on its benzene ring (W¹) as previously described. Accordingly, there are included compounds in which Ar—V¹—W¹ is a 3-(benzamido)phenyl group substituted on the benzene ring of the benzamido moiety by lower alkyl or lower alkoxy, wherein the alkyl group or the alkyl part of the alkoxy group is optionally substituted by at least one halogen, e.g. F; such substitution by F is preferred in one embodiment.

One class of especially preferred Ar—V¹—W¹ groups comprise 3-(benzamido) phenyl groups the benzene ring of whose benzamido moiety is substituted by a fluorinated moiety, particularly substituted 3-(benzamido)phenyl groups whose benzamido moiety is substituted by fluoroalkoxy. A particularly preferred Ar—V¹—W¹, group comprises a phenyl-3-(1,1,2,2-tetrafluoroethoxy)benzamide group.

Any reference to compounds, salts and the like in the plural is always to be understood as including one compound, one salt or the like.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

In a preferred embodiment of the present invention, each R¹ and R², when present, is hydrogen, thus giving a preferred general formula II:

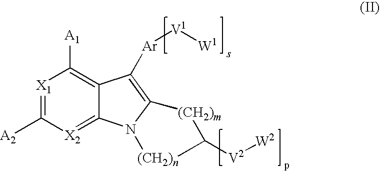

where all atoms and groups are as hereinbefore described for formula I.

Further preferred formulae derived from formulae I and II are shown below, in formulae III, IV, V, VI, VII and VIII. All atoms and groups are as hereinbefore described, including preferences thereof where appropriate.

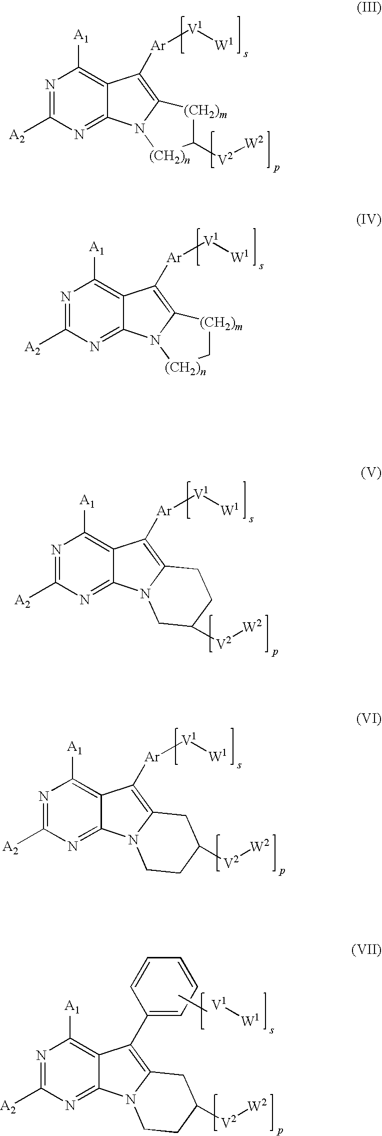

In the compounds of formula (VII), A₁ and A₂ are often selected from H and NR^{a}R^{b}; in a sub-class, one (often A₁) is NR^{a}R^{b} and the other is H; often R^{a} and R^{b} are each H or lower alkyl, e.g. both may be H. In the compounds of the invention, including those of formulae (I), (II), (III), (IV), (V), (VI) and (VII), Ar—(V¹—W¹)ₛ is desirably Ph-(V¹—W¹)ₛ. One preferred class of compounds having this structure is of formula (VIII).

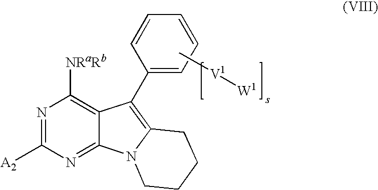

In formula (VIII), R^{a} and R^{b} are suitably both H, but sometimes one or both are lower alkyl, for example. A² is suitably H or NR^{a}R^{b}, where R^{a} and R^{b} are suitably both H; usually, A² is H.

As previously stated, s is preferably 1, including in the case of compounds of formulae (I), (II), (III), (IV), (V), (VI,) (VII) and (VIII).

Preferred compounds of the invention, including those of formulae (I), (II), (III), (IV), (V), (VI,) (VII) and (VIII), have an Ar—(V¹—W¹)ₛ group of the structure (IX):

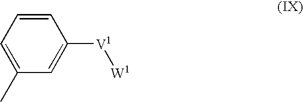

Included are compounds in which structure (IX) contains one or more further substituents as described above for substituents of moiety Ar, for example 1 or 2 substituents selected from halo (e.g. F), lower alkyl, lower alkoxy, amino or hydroxy.

An exemplary W¹ group, including in the case of compounds of formulae (I), (II), (III), (IV), (V), (VI,) (VII) and (VIII), as well as all those compounds having an Ar—(V¹—W¹)ₛ group of formula (IX), is of formula (X):

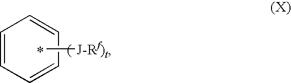

where J is selected from O, NR^{a}, S, hydrocarbyl (e.g. lower alkyl), halohydrocarbyl (e.g. lower alkyl substituted one or more times by F) or a covalent bond;
R^{f} is selected from halo, H, NR^{a}R^{b}, OR^{c}, SR^{c},
where R^{a}, R^{b} and R^{c} are as hereinbefore described and independent of each other; and t is 0, 1, 2, 3 or 4.

In a particularly preferred embodiment, the present invention relates to compounds of formula (X), or pharmaceutically acceptable salts, esters, prodrugs or N-oxides thereof:

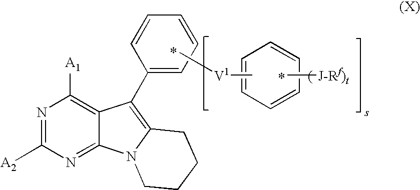

Where the symbols have the meanings previously ascribed to them, for example), A₁ and A₂ may be as previously described with reference to formula (VII).

It is preferred that the compound comprises a V¹-Ph-(J-R^{f})ₜ moiety attached to the phenyl ring in the meta position.

It is preferred that s is 1. An aspect of the invention resides in N-[3-(1-Amino-5,6,7,8-tetrahydro-2,4,4b-triazafluoren-9-yl)-phenyl]benzamides, whose benzamide moiety is optionally substituted one or more times on its benzene ring, e.g. by a J-R^{f} group as hereinbefore described.

Salts are especially the pharmaceutically acceptable acid addition salts of active compounds of the invention, including those of formula I. Such salts are formed, for example, by compounds of formula I having a basic nitrogen atom as acid addition salts, preferably with organic or inorganic acids, especially the pharmaceutically acceptable salts. Suitable inorganic acids are, for example, hydrohalic acids, such as hydrochloric acid; sulfuric acid; or phosphoric acid. Suitable organic acids are, for example, carboxylic, phosphonic, sulfonic or sulfamic acids, for example acetic (ethanoic) acid, propionic (propanoic) acid, octanoic acid, decanoic acid, dodecanoic acid, glycolic acid, lactic acid, 2-hydroxybutyric acid, gluconic acid, glucosemonocarboxylic acid, fumaric acid, succinic acid, adipic acid, pimelic acid, suberic acid, azelaic acid, malic acid, tartaric acid, citric acid, glucaric acid, galactaric acid, amino acids, such as glutamic acid, aspartic acid, N-methylglycine, acetylaminoacetic acid, N-acetylasparagine, N-acetylcysteine, pyruvic acid, acetoacetic acid, phosphoserine, 2- or 3-glycerophosphoric acid, maleic acid, hydroxymaleic acid, methylmaleic acid, cyclohexanecarboxylic acid, benzoic acid, salicylic acid, 1- or 3-hydroxynaphthyl-2-carboxylic acid, 3,4,5-trimethoxybenzoic acid, 2-phenoxybenzoic acid, 2-acetoxybenzoic acid, 4-aminosalicylic acid, phthalic acid, phenylacetic acid, glucuronic acid, galacturonic acid, methane- or ethane-sulfonic acid, 2-hydroxyethanesulfonic acid, ethane-1,2-disulfonic acid, benzenesulfonic acid, 1,5-naphthalenedisulfonic acid, 2-naphthalenesulfonic acid, N-cyclohexylsulfamic acid, N-methyl-, N-ethyl- or N-propyl-sulfamic acid, or other organic protonic acids, such as ascorbic acid.

For isolation or purification it is also possible to use pharmaceutically unacceptable salts, for example picrates or perchlorates. Only the pharmaceutically acceptable salts or the free compounds (optionally in the form of pharmaceutical compositions) are used therapeutically, and those are therefore preferred.

In view of the close relationship between the novel compounds in free form and in the form of their salts, including also those salts which can be used as intermediates, for example in the purification of the novel compounds or for their identification, hereinbefore and hereinafter any reference to the free compounds is also to be understood as including the corresponding salts, as appropriate and expedient.

The compounds (I) of the present invention are found to inhibit, regulate and/or modulate tyrosine and serine/threonine kinase and kinase-like proteins involved in signal transduction, and compositions containing the compounds are used in the treatment of tyrosine and serine/threonine kinase and kinase-like-dependent diseases, such as angiogenesis, cancer, tumour growth, atherosclerosis, age related macular degeneration, diabetic retinopathy, inflammatory diseases, neurotraumatic diseases, chronic neurodegeneration, pain, migraine or cardiac hypertrophy, and the like in mammals.

Specifically, the compounds (I) of the present invention inhibit PDGF-R, Kdr, c-Src, Her-1, Her-2, c-Kit, c-Abl, Ins-r, Tek, Flt-1, Flt-3, Flt-4, c-Abi and FGFR-1 at >70% inhibition at 10 micromole. More specifically, the compounds inhibit the RAF family of kinases, including mutant RAF family kinase members, with IC50 values in the range of 50-1000 nM.

Typically, the patient is a mammal, generally a human, suffering from a disease that is characterized by excessive signaling through the MAP kinase pathway. This can be measured by activation state specific antibodies to pathway members by methods such as Western blot analysis or immunohistochemistry. Such methods are known to those of skill in the art.

In general, the disease characterized by excessive signaling through the MAP kinase signaling pathway is a proliferative disease, particularly a cancer characterized by increased RAF kinase activity, for example one which overexpresses wild-type B- or C-RAF kinase, or that expresses an activating mutant RAF kinase, for example a mutant B-RAF kinase. Cancers wherein a mutated RAF kinase has been detected include melanoma, colorectal cancer, ovarian cancer, gliomas, adenocarcinomas, sarcomas, breast cancer and liver cancer. Mutated B-RAF kinase is especially prevalent in many melanomas.

In accordance with the present invention, a sample of diseased tissue may taken from the patient, for example, as a result of a biopsy or resection, and tested to determine whether the tissue produces a mutant RAF kinase, such as a mutant B-RAF kinase or overexpresses a wild-type RAF kinase, such as wild-type B- or C-RAF kinase. If the test indicates that mutant RAF kinase is produced or that a RAF kinase is overproduced in the diseased tissue, the patient is treated by administration of an effective RAF-inhibiting amount of a RAF inhibitor compound described herein.

However, it is also possible to downregulate the MAP kinase signaling pathway with a RAF kinase inhibiting compound If another kinase in the cascade is the cause of excessive signaling in the pathway. Thus, the present invention further relates to the treatment of a disease characterized by excessive signaling in the MAP kinase signaling pathway attributed to a cause other than an activating mutation in or overexpression of a RAF kinase.

Tissue samples are tested by methods generally known in the art. For example, B-RAF mutations are detected by allele specific PCR, DHPLC, mass spectroscopy and overexpression of wild-type B- or C-RAF detected by immunohistochemistry, immunofluorescence, or Western blot analysis. A particularly useful method of detecting B-RAF mutations is a polymerase chain reaction based method. Similar methods are used to determine whether other kinases in the cascade are mutant or overexpressed.

A particularly important aspect of this invention relates to a method of treating melanoma, which comprises (a) testing melanoma tissue from a patient to determine whether the melanoma tissue expresses mutant RAF kinase or overexpresses a wild-type RAF kinase and (b) treating the patient with an effective RAF kinase inhibiting amount of a RAF-inhibiting compound described herein if the melanoma tissue is found to overexpress a wild type RAF kinase or express an activating mutant B-RAF kinase.

An important aspect of this embodiment relates to a method of treating melanoma, which comprises (a) testing melanoma tissue from a patient to determine whether the melanoma tissue overexpresses B-RAF kinase or C-RAF kinase activity and (b) treating the patient with an effective RAF kinase inhibiting amount of a RAF inhibiting compound described herein if the melanoma tissue is found to overexpress the B-RAF kinase or C-RAF kinase activity.

Another important aspect of this embodiment relates to a method of treating melanoma, which comprises (a) testing melanoma tissue from a patient to determine whether the melanoma tissue expresses mutant B-RAF kinase and (b) treating the patient with an effective RAF kinase inhibiting amount of a RAF inhibiting compound described herein if the melanoma tissue is found to express mutant B-RAF kinase.

Generally, the B-RAF kinase mutation is one of those described in the Davies et al article cited. These mutations are summarized in Table 1.

TABLE 1

| B-RAF mutation | protein change |
|---|---|
| G1388A | G463E |
| G1388T | G463V |
| G1394C | G465A |
| G1394A | G465E |
| G1394T | G465V |
| G1403C | G468A |
| G1403A | G468E |
| G1753A | E585K |
| T1782G | F594L |
| G1783C | G595R |
| C1786G | L596V |
| T1787G | L596R |
| T1796A | V599E |
| TG1796-97AT | V599D |

Thus, the present invention particularly relates to a method of treating a disease characterized by an activated mutant B-RAF kinase, which comprises detecting a mutation in the B-RAF kinase gene or protein in a tissue sample from a patient and treating the patient with an effective B-RAF kinase inhibiting compound, especially a compound described herein.

Hence, the present invention additionally relates to a compound (I) for use in the treatment of melanoma. More particularly, the invention relates to a compound (I) for use in the treatment of a disease characterized by an activated mutant B-RAF kinase.

Further, the invention provides for the use of a compound (I) in the manufacture of a medicament for use in the treatment of melanoma. More specifically, the invention provides for the use of a compound (I) in the manufacture of a medicament for use in the treatment of a disease characterized by an activated mutant B-RAF kinase.

An important aspect of this invention includes those instances wherein the mutant B-RAF kinase exhibits a mutation described in Table 1, especially the V599E mutation.

A particularly important aspect of this invention includes those instances wherein disease is melanoma and the mutant B-RAF kinase exhibits a mutation described in Table 1, especially the V599E mutation.

Accordingly, this invention includes a method of treating a disease characterized by mutant B-RAF kinase, which comprises detecting a mutation in the B-RAF kinase gene selected from G1388A, G1388T, G1394C, G1394A, G1394T, G1403C, G1403A, G1753A, T1782G, G1783C, C1786G, T1787G, T1796A and TG1796-97AT, or corresponding mutation in the RAF kinase protein, in a tissue sample from a patient and treating the patient with an effective B-RAF kinase inhibiting compound described herein.

The present invention further relates to a method of inhibiting RAF kinase, which comprises contacting the RAF kinase with a compound of formula (I). Preferably, the RAF kinase is B- or C-RAF kinase, or a mutant RAF kinase, especially a mutant B-RAF kinase, particularly the V599E mutant. The RAF kinase may be isolated or in a cellular environment.

The compounds of formula I have valuable pharmacological properties, as described above.

The compounds of the present invention may be administered alone or in combination with other anticancer agents, such as compounds that inhibit tumor angiogenesis, for example, the protease inhibitors, epidermal growth factor receptor kinase inhibitors, vascular endothelial growth factor receptor kinase inhibitors and the like; cytotoxic drugs, such as antimetabolites, like purine and pyrimidine analog antimetabolites; antimitotic agents like microtubule stabilizing drugs and antimitotic alkaloids; platinum coordination complexes; anti-tumor antibiotics; alkylating agents, such as nitrogen mustards and nitrosoureas; endocrine agents, such as adrenocorticosteroids, androgens, anti-androgens, estrogens, anti-estrogens, aromatase inhibitors, gonadotropin-releasing hormone agonists and somatostatin analogues and compounds that target an enzyme or receptor that is overexpressed and/or otherwise involved a specific metabolic pathway that is upregulated in the tumor cell, for example ATP and GTP phosphodiesterase inhibitors, protein kinase inhibitors, such as serine, threonine and tyrosine kinase inhibitors, for example, Abelson protein tryosine kinase and the various growth factors, their receptors and kinase inhibitors therefore, such as, epidermal growth factor receptor kinase inhibitors, vascular endothelial growth factor receptor kinase inhibitors, fibroblast growth factor inhibitors, insulin-like growth factor receptor inhibitors and platelet-derived growth factor receptor kinase inhibitors and the like; methionine aminopeptidase inhibitors, proteasome inhibitors, cyclooxygenase inhibitors, for example, cyclooxygenase-1 or -2 inhibitors, and histone deacetylase inhibitors.

The compound of the present invention may also be administered together with radiotherapy, immunotherapy, surgical treatment or combinations thereof. Treatment to maintain the status of a patient after tumor remission or even chemopreventive treatment, for example in the case of at-risk patients, is also possible.

Compounds according to the invention are intended not only for the (prophylactic and, preferably, therapeutic) treatment of human beings, but also for the treatment of other warm-blooded animals, for example of commercially useful animals, for example rodents, such as mice, rabbits or rats, or guinea pigs.

In general, the invention relates also to the use of a compound of formula I in inhibiting RAF kinase activity.

The compounds of the present invention are preferably administered as an active ingredient in a pharmaceutical composition. Preference is given to a pharmaceutical composition which is suitable for administration to a warm-blooded animal, especially a human being or a commercially useful mammal, which is suffering from a disease characterized by an aberrant MAP kinase signaling pathway especially, a tumor disease, most particularly melanoma, comprising a compound of formula I, or a pharmaceutically acceptable salt thereof where salt-forming groups are present, in an amount that is effective in inhibiting RAF kinase, particularly a mutant RAF kinase, together with at least one pharmaceutically acceptable carrier.

Preference is given also to a pharmaceutical composition for the prophylactic or, especially, therapeutic treatment of tumor diseases and other proliferative diseases in a warm-blooded animal, especially a human being or a commercially useful mammal, which requires such treatment, especially which is suffering from such a disease, comprising a novel compound of formula I, or a pharmaceutically acceptable salt thereof, as active ingredient in an amount that is effective prophylactically or, especially, therapeutically against the mentioned diseases.

Pharmaceutical compositions comprise from approximately 1% to approximately 95% active ingredient, dosage forms that are in single dose form preferably comprising from approximately 20% to approximately 90% active ingredient, and dosage forms that are not in single dose form preferably comprising from approximately 5% to approximately 20% active ingredient. Unit dose forms are, for example, dragées, tablets, ampoules, vials, suppositories or capsules. Other dosage forms are, for example, ointments, creams, pastes, foams, tinctures, lipsticks, drops, sprays, dispersions, etc. Examples are capsules comprising from approximately 0.05 g to approximately 1.0 g of the active ingredient.

The pharmaceutical compositions of the present invention are prepared in a manner known per se, for example by means of conventional mixing, granulating, confectioning, dissolving or lyophilising processes.

Solutions of the active ingredient are preferably used, in addition also suspensions or dispersions, especially isotonic aqueous solutions, dispersions or suspensions, which, in the case of, for example, lyophilised compositions which contain the active substance alone or together with a carrier, for example mannitol, can be prepared prior to use. The pharmaceutical compositions may be sterilised and/or comprise excipients, for example preservatives, stabilisers, wetting agents and/or emulsifiers, solubilisers, salts for regulating the osmotic pressure and/or buffers, and are prepared in a manner known per se, for example by means of conventional dissolving or lyophilising processes. The mentioned solutions or suspensions may comprise viscosity-increasing substances, such as sodium carboxymethylcellulose, carboxymethylcellulose, dextran, polyvinylpyrrolidone or gelatin, or solubilisers, for example Tween 80 [polyoxyethylene(20)sorbitan monooleate; trade mark of ICI Americas, Inc, USA].

Suspensions in oil comprise as the oily component the vegetable, synthetic or semi-synthetic oils customary for injection purposes. There may be mentioned as such especially liquid fatty acid esters, which comprise as the acid component a long-chained fatty acid having from 8 to 22, especially from 12 to 22, carbon atoms, for example lauric acid, tridecylic acid, myristic acid, pentadecylic acid, palmitic acid, margaric acid, stearic acid, arachidic acid, behenic acid or corresponding unsaturated acids, for example oleic acid, elaidic acid, erucic acid, brassidic acid or linoleic acid, optionally with the addition of anti-oxidants, for example vitamin E, β-carotene or 3,5-di-tert-butyl-4-hydroxytoluene. The alcohol component of those fatty acid esters has a maximum of 6 carbon atoms and is a mono- or poly-hydric, for example mono-, di- or tri-hydric, alcohol, for example methanol, ethanol, propanol, butanol or pentanol or their isomers, but especially glycol and glycerol. Examples of fatty acid esters which may be mentioned are, therefore: ethyl oleate, isopropyl myristate, isopropyl palmitate, “Labrafil M 2375” (polyoxyethyleneglycerol trioleate from Gattefossé, Paris), “Labrafil M 1944 CS” (unsaturated polyglycolized glycerides prepared by alcoholysis of apricot kernel oil and composed of glycerides and polyethylene glycol ester; Gattefossé, France), “Labrasol” (saturated polyglycolized glycerides prepared by alcoholysis of TCM and composed of glycerides and polyethylene glycol ester; Gattefossé, France) and/or “Miglyol 812” (triglyceride of saturated fatty acids having a chain length of from C₈ to C₁₂ from Hüls AG, Germany), but especially vegetable oils, such as cottonseed oil, almond oil, olive oil, castor oil, sesame oil, soybean oil and, more especially, groundnut oil.

The preparation of the injection compositions is carried out in customary manner under sterile conditions, as are also the introduction thereof, for example, into ampoules or vials and the sealing of the containers.

Pharmaceutical compositions for oral administration can be obtained, for example, by combining the active ingredient with one or more solid carriers, granulating a resulting mixture, where appropriate, and processing the mixture or granules, if desired, where appropriate by addition of additional excipients, to tablets or dragée cores.

Suitable carriers are especially fillers, such as sugars, for example lactose, saccharose, mannitol or sorbitol, cellulose preparations and/or calcium phosphates, for example tricalcium phosphate or calcium hydrogen phosphate, also binders, such as starches, for example corn, wheat, rice or potato starch, methylcellulose, hydroxypropylmethylcellulose, sodium carboxymethylcellulose and/or polyvinylpyrrolidone, and/or, if desired, disintegrators, such as the above-mentioned starches, also carboxymethyl starch, crosslinked polyvinylpyrrolidone, alginic acid or a salt thereof, such as sodium alginate. Additional excipients are especially flow conditioners and lubricants, for example silicic acid, talc, stearic acid or salts thereof, such as magnesium or calcium stearate, and/or polyethylene glycol, or derivatives thereof.

Dragée cores can be provided with suitable, optionally enteric, coatings, there being used inter alia concentrated sugar solutions which may contain gum arabic, talc, polyvinylpyrrolidone, polyethylene glycol and/or titanium dioxide, or coating solutions in suitable organic solvents or solvent mixtures or, for the preparation of enteric coatings, solutions of suitable cellulose preparations, such as acetylcellulose phthalate or hydroxypropylmethylcellulose phthalate. Colorings or pigments may be added to the tablets or dragée coatings, for example for identification purposes or to indicate different doses of active ingredient.

Pharmaceutical compositions for oral administration are also hard gelatin capsules and soft sealed capsules consisting of gelatin and a plasticiser, such as glycerol or sorbitol. The hard gelatin capsules may contain the active ingredient in the form of granules, for example in admixture with fillers, such as corn starch, binders and/or glidants, such as talc or magnesium stearate, and optionally stabilisers. In soft capsules the active ingredient is preferably dissolved or suspended in suitable liquid excipients, such as fatty oils, paraffin oil or liquid polyethylene glycols or fatty acid esters of ethylene glycol or propylene glycol, it likewise being possible to add stabilisers and detergents, for example of the polyoxyethylenesorbitan fatty acid ester type.

Suitable rectally administrable pharmaceutical compositions are, for example, suppositories that consist of a combination of the active ingredient with a suppository base. Suitable suppository bases are, for example, natural or synthetic triglycerides, paraffin hydrocarbons, polyethylene glycols or higher alkanols.

For parenteral administration there are suitable, especially, aqueous solutions of an active ingredient in water-soluble form, for example in the form of a water-soluble salt, or aqueous injection suspensions that comprise viscosity-increasing substances, for example sodium carboxymethylcellulose, sorbitol and/or dextran and, if desired, stabilisers. The active ingredient, optionally together with excipients, can also be in the form of a lyophilisate and can be made into a solution prior to parenteral administration by the addition of suitable solvents.

Solutions used, for example, for parenteral administration can also be used as infusion solutions.

Preferred preservatives are, for example, antioxidants, such as ascorbic acid, or microbicides, such sorbic acid or benzoic acid.

The invention relates especially to a process or a method for treating one of the pathological conditions that is characterized by an aberrant MAP kinase signaling pathway, especially a disease responsive to inhibition of RAF kinase, especially a corresponding tumor disease. The compounds of formula I can be administered prophylactically or therapeutically as such or in the form of pharmaceutical compositions, preferably in an amount that is effective against the mentioned diseases, to a warm-blooded animal, for example a human being, requiring such treatment, the compounds being used especially in the form of pharmaceutical compositions. In the case of a body weight of approximately 70 kg, a daily dose of from approximately 0.1 g to approximately 5 g, preferably from approximately 0.5 g to approximately 2 g, of a compound of the present invention is administered.

The preferred dosage, composition and preparation of pharmaceutical formulations (medicaments) to be used in each particular case are described above.

The compounds of the present invention are prepared utilizing methods known to those of ordinary skill in the art according to the exemplary reaction scheme described below.

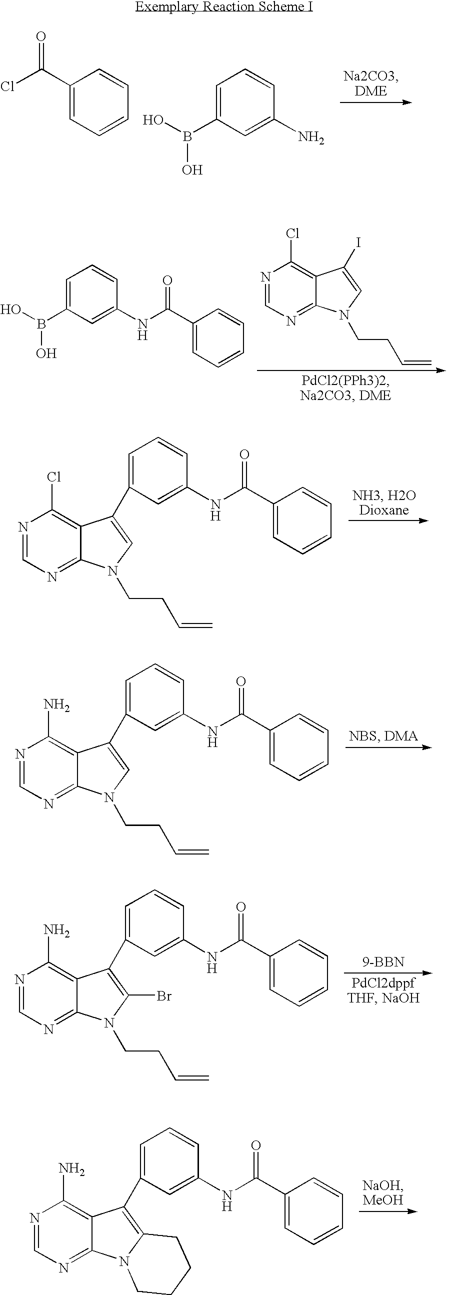

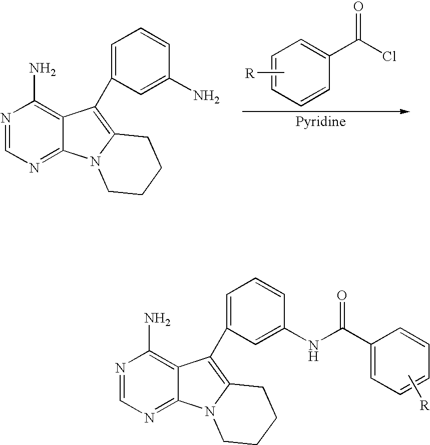

Included in the invention therefore are intermediates of the formula

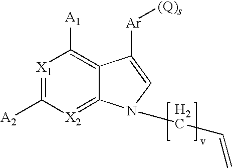

wherein
Q is a group of the formula V¹—W¹ or a moiety comprising an optionally protected functional group capable of being converted to a V¹—W¹ group, as for example in the case of a protected amine capable of being converted, after deprotection, to an amide linker bonded to
a V¹ moiety;
v is from 1 to 9, e.g. 2 or 3; and
all other symbols are as described previously.

The preparative method will now be illustrated by reference to the following specific preparation of N-[3-(1-amino-5,6,7,8-tetrahydro-2,4,4b-triazafluoren-9-yl)-phenyl]-3-(1,1,2,2-tetrafluoro-ethoxy)benzamide.

### 4-Chloro-5-iodo-7H-pyrrolo[2,3-D]pyrimidine

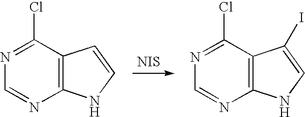

To a solution of 4-chloro-7H-pyrrolo[2,3-D]pyrimidine¹ (39 mmol) in DMF (50 mL) was added in several portions N-iodosuccinimide (8.8 g). After stirring overnight at 20° C., EtOAc (500 ml) was added and the solution was washed three times with water (150 ml). The organic layer was filtered through a short silica column and concentrated in vacuo. Yield: 86% (9.3 g); MS: 279 ¹ Available from Toronto Research Chemicals.

### 7-But-3-enyl-4-chloro-5-iodo-7H-pyrrolo[2,3-Dipyrimidine

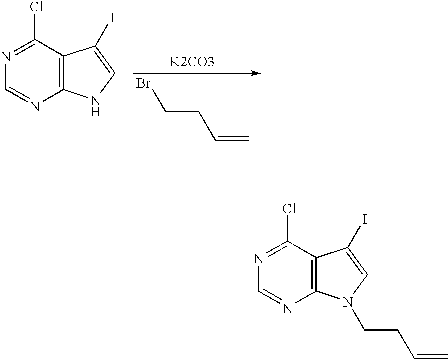

To a solution of 4-chloro-5-iodo-7H-pyrrolo[2,3-D]pyrimidine (32.2 mmol) in DMF (50 ml) was added potassium carbonate (39 mmol) followed by 4-bromo-1-butene (39 mmol). After stirring at 20° C. overnight, EtOAc (500 ml) was then added and the solution was washed three times with water (150 ml). The organic layer was filtered through a short silica column and concentrated in vacuo. Yield: 80% (8.5 g); MS: 333

### N-(3-Phenylboronic acid)-benzamide

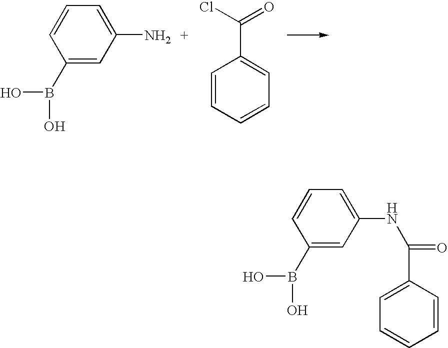

To a sealed tube containing 3-aminophenylboronic acid (9 mmol), sodium carbonate (21 mmol) in DME (50 mL) was added at 0° C. benzoyl chloride (14 mmol). After warming to 20° C. for 0.3 h, water (50 ml) was added and stirring was continued for another 0.3 h. The resulting solution was immediately used in the next step.

### N-[3-(7-But-3-enyl-4-chloro-7H-pyrrolo[2,3-d]pyrimidin-5-yl)-phenyl]-benzamide

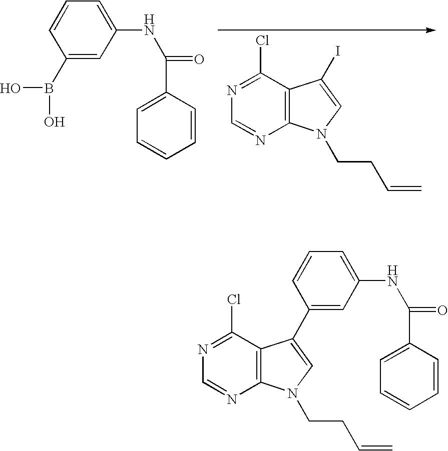

To the above solution of boronic acid was added 7-but-3-enyl-4-chloro-5-iodo-7H-pyrrolo[2,3-D]pyrimidine (7.2 mmol) and dichlorobis(triphenylphosphine)palladium (0.85 mmol). The solution was flushed with nitrogen, sealed, and heated at 80° C. for 2 h. After cooling the solution was extracted three times with DCM (100 ml), dried over sodium sulfate, and concentrated in vacuo. The product was obtained from purification on silica gel. Yield: 41% (1.2 g); MS: 403

### N-[3-(4-Amino-7-but-3-enyl-7H-pyrrolo[2,3-D]pyrimidin-5-yl)-phenyl]-benzamide

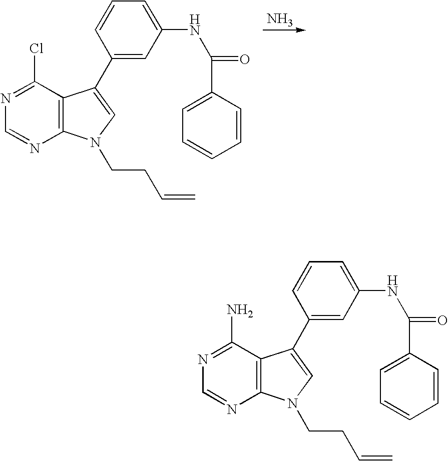

To a sealed tube containing ammonia hydroxide (33%, 20 mL) and dioxane (20 mL) was added N-[3-(7-but-3-enyl-4-chloro-7H-pyrrolo[2,3-D]pyrimidin-5-yl)-phenyl]-benzamide (3 mmol). The tube was heated at 120° C. for 16 h and then concentrated in vacuo. The residue was dissolved in DCM (2×80 ml), filtered through Mg₂SO₄ and concentrated in vacuo. Yield: 78% (0.9 g); MS: 383.

### N-[3-(4-Amino-6-bromo-7-but-3-enyl-7H-pyrrolo[2,3-D]pyrimidin-5-yl)-phenyl]-benzamide

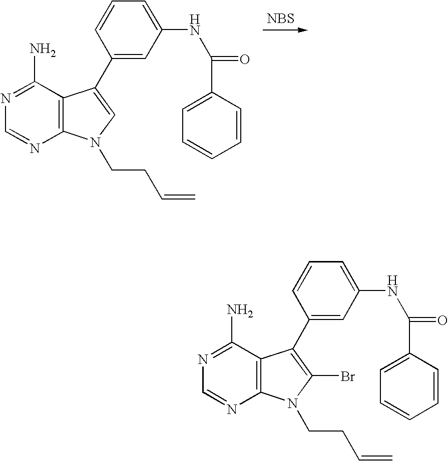

To a solution of N-[3-(4-amino-7-but-3-enyl-7H-pyrrolo[2,3-D]pyrimidin-5-yl)-phenyl]-benzamide (2.3 mmol) in DMF (10 mL) was added in several portions NBS (2.3 mmol). After stirring for 10 m, EtOAc (150 ml) was added. The solution washed with twice with water (30 ml), dried over MgSO₄ and concentrated in vacuo. Yield: 90% (1 g); MS: 462.

### N-[3-(1-Amino-5,6,7,8-tetrahydro-2,4,4b-triazafluoren-9-yl)-phenyl]benzamide

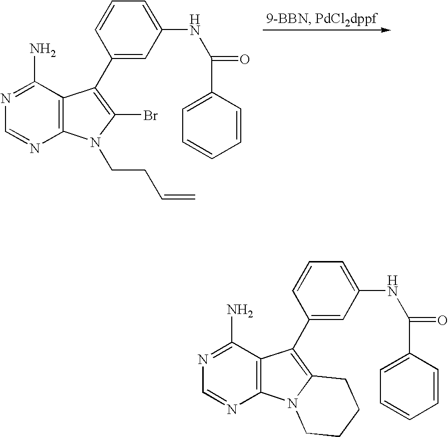

In a sealed tube containing 9-BBN solution (0.5M, 25 ml) at 0° C. was added N-[3-(1-amino-5,6,7,8-tetrahydro-2,4,4b-triazafluoren-9-yl)-phenyl]benzamide (2.16 mmol). After warming to 20° C. and stirring for 5 h, NaOH (3M, 10 ml) was added dropwise, followed by PdCl₂dppf (340 mg). The tube was flushed with N₂, sealed and heated to 80° C. for 15 h. After cooling the solution was concentration in vacuo and the product was obtained from purification on silica gel. Yield: 33% (280 mg); MS: 383

### 9-(3-Amino-phenyl)-5,6,7,8-tetrahydro-2,4,4b-triazafluoren-1-ylamine

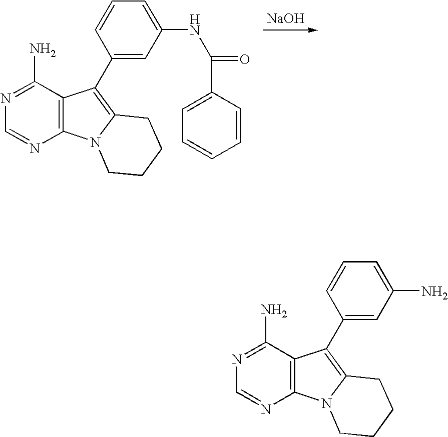

In a sealed tube containing 10 M NaOH (8 mL) and MeOH (8 mL) was added N-[3-(1-amino-5,6,7,8-tetrahydro-2,4,4b-triazafluoren-9-yl)-phenyl]benzamide (0.75 mmol). After heating at 80° C. for 6 h, the solution was cooled to 20° C. and concentrated in vacuo. The resulting white precipitate was obtained by filtration and air dried. Yield: 80% (166 mg); MS: 279

### N-[3-(1-Amino-5,6,7,8-tetrahydro-2,4,4b-triazafluoren-9-yl)-phenyl]-3-(1,1,2,2-tetrafluoro-ethoxy)benzamide

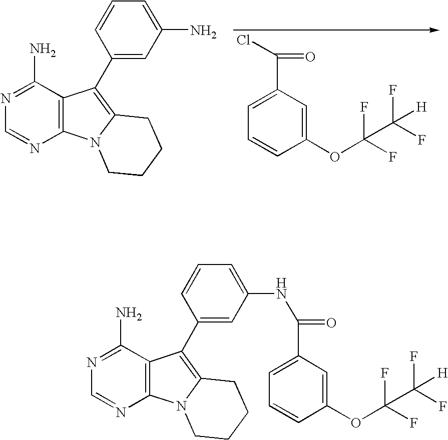

To a solution of 9-(3-aminophenyl)-5,6,7,8-tetrahydro-2,4,4b-triazafluoren-1-ylamine (0.6 mmol) in pyridine (5 mL) and DCM (2 mL) was added 3-(1,1,2,2-tetrafluoroethoxy)benzoyl chloride² (0.6 mmol). After 10 m, the solution was concentrated in vacuo. The product was obtained after purification silica gel. Yield: 53% (160 mg); MS: 499.

Synthesized involving 3-(1,1,2,2-tetrafluoroethoxy)benzoic acid and thionyl chloride is also contemplated, using obvious modifications of the process shown above.

1. A compound of formula (I) or a pharmaceutically acceptable salt, ester, prodrug or N-oxide thereof: 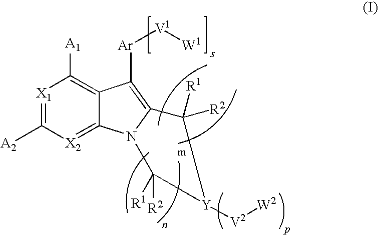
wherein
A₁ and A₂ are each independently selected from H, NR^{a}R^{b}, OR^{c}, SR^{c} or alkyl e.g. lower alkyl or aryl,
where R^{a} and R^{b} are each independently selected from hydrogen; OH; hydrocarbyl and hydrocarbyloxy, the hydrocarbyl moieties optionally being substituted by one or more substituents selected from halo and hydroxy; mercapto; guanidine; NH₂; NHR^{d}; N(R^{d})₂;
where R^{d} is hydroxy or alkyl;
where R^{c} is selected from hydrogen and hydrocarbyl, the hydrocarbyl optionally being substituted by one or more substituents selected from halo and hydroxy;
X₁ and X₂ are each independently selected from N or CR^{c};
m, n and s are each independently selected from 0, 1, 2, 3, 4,5; and
p is 0 or 1, such that p+s≧1;
Y is selected from O, S, N or C and, where Y is O or S, p=0;
V¹ and V² are each independently a linker, the linker being a group consisting of one or more of lower alkyl, amine, ether, amide, ester, urea, carbamate, sulphonamide or are a direct bond;
W¹ and W² are each independently selected from H, alkyl, or a substituted or unsubstituted cyclic group;
Ar is a substituted or unsubstituted aryl group selected from phenyl, napthyl, oxiranyl, azirinyl, 1,2-oxathiolanyl, imidazolyl, thienyl, furyl, tetrahydrofuryl, pyranyl, thiopyranyl, thianthrenyl, isobenzofuranyl, benzofuranyl, chromenyl, 2H-pyrrolyl, pyrrolyl, pyrrolinyl, pyrrolidinyl, imidazolyl, imidazolidinyl, benzimidazolyl, pyrazolyl, pyrazinyl, pyrazolidinyl, pyranyol, thiazolyl, isothiazolyl, dithiazolyl, oxazolyl, isoxazolyl, pyridyl, pyrazinyl, pyrimidinyl, piperidyl, piperazinyl, pyridazinyl, morpholinyl, thiomorpholinyl, indolizinyl, isoindolyl, 3H-indolyl, indolyl, benzimidazolyl, cumaryl, indazolyl, triazolyl, tetrazolyl, purinyl, 4H-quinolizinyl, isoquinolyl, quinolyl, tetrahydroquinolyl, tetrahydroisoquinolyl, decahydroquinolyl, octahydroisoquinolyl, benzofuranyl, dibenzofuranyl, benzothiophenyl, dibenzothiophenyl, phthalazinyl, naphthyridinyl, quinoxaloyl, quinazolinyl, quinazolinyl, cinnolinyl, pteridinyl, carbazolyl, β-carbolinyl, phenanthridinyl, acridinyl, perimidinyl, phenanthrolinyl, furazanyl, phenazinyl, phenothiazinyl, phenoxazinyl, chromenyl, isochromanyl and chromanyl;
each R¹ and R², if present, are independently selected from hydrogen, lower-alkyl, halo and hydroxyl, wherein when n or m is ≦1, each R¹ and R² may be the same or different.

2. A compound of claim 1 wherein A₁ and A₂ are each the same or different and an NR^{a}R^{b} group.

3. A compound of claim 1 wherein at least one of A₁ and A₂ is NR^{a}R^{b}.

4. A compound of claim 1 wherein one of A₁ and A₂ is NH₂ and the other of A₁ and A₂ is H.

5. A compound of claim 1 wherein X₁ and X₂ are N.

6. A compound of claim 1 wherein m+n=2 or 3.

7. A compound of claim 1 wherein m+n=3.

8. A compound of claim 1 wherein Y is C.

9. A compound of claim 1 wherein V² is a direct bond.

10. A compound of claim 1 wherein W² is H.

11. A compound of claim 1 wherein s is at least 1.

12. A compound of claim 1 wherein Ar is a phenyl group.

13. A compound of claim 1 wherein W¹ is a substituted phenyl group.

14. A compound of claim 1 wherein W¹ has the formula X 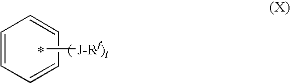
where J is selected from O, NR^{a}, S, hydrocarbyl, halohydrocarbyl or a covalent bond;
R^{f} is selected from halo, H, NR^{a}R^{b}, OR^{c}, SR^{c}; and
t is 0, 1, 2, 3 or 4.

15. A compound of claim 1 wherein s is 1.

16. A compound of claim 1 wherein V¹ is an amide linker.

17. A compound of claim 1 wherein Ar—V¹—W¹ is a benzamido-phenyl group.

18. A compound of claim 1 wherein Ar—V¹—W¹ is a 3-(benzamido)phenyl group.

19. A compound of claim 1 wherein W¹ comprises at least one halogen-containing group.

20. A compound of claim 18 wherein W¹ comprises at least one fluoroalkoxy group.

21. A compound of claim 1 wherein W¹ comprises at least one fluoro lower alkoxy group.

22. A compound of claim 1 wherein Ar—V¹—W¹ is a 3-(benzamido)phenyl group substituted on the benzene ring of the benzamido moiety by lower alkyl or lower alkoxy, wherein the alkyl group or the alkyl part of the alkoxy group is optionally substituted by at least one halogen.

23. A compound of claim 1 wherein Ar—V¹—W¹ is a 3-(benzamido)phenyl group substituted on the benzene ring of the benzamido moiety by fluoroalkoxy.

24. A compound of claim 1 wherein Ar—V¹—W¹ is a phenyl-3-(1,1,2,2-tetrafluoroethoxy)benzamide group.

25. A compound of claim 1 wherein each R¹ and R² is hydrogen.

26. A compound of claim 14, wherein a N-[3-(1-amino-5,6,7,8-tetrahydro-2,4,4b-triazafluoren-9-yl)-phenyl]benzamide, whose benzamide moiety is optionally substituted one or more times on its benzene ring by a J-R^{f} group.

27. A compound of claim 1 for use as a pharmaceutical.

28. A compound of claim 27 for use in the treatment of in the treatment of a tyrosine or serine/threonine kinase or kinase-like-dependent disease.

29. A compound of claim 1 for use in the treatment of melanoma, angiogenesis, cancer, tumour growth, atherosclerosis, age related macular degeneration, diabetic retinopathy, inflammatory diseases, neurotraumatic diseases, chronic neurodegeneration, pain, migraine or cardiac hypertrophy.

30. A compound of claim 1 for use in the treatment of a disease characterized by an activated mutant B-RAF kinase.

31. (canceled)

32. (canceled)

33. (canceled)

34. (canceled)

35. (canceled)

36. (canceled)

37. (canceled)

38. A pharmaceutical composition comprising a compound of claim 1.

39. A pharmaceutical composition as comprising from approximately 1% to approximately 95% of a compound of claim 1.

40. A pharmaceutical composition comprising from approximately 20% to approximately 90% of a compound of claim 1.

41. A pharmaceutical composition comprising from approximately 5% to approximately 20% of a compound claim 1.

42. A pharmaceutical composition of compound I of claim 1 for administration by injection.

43. A pharmaceutical composition of claim 42 comprising a solution, suspension or dispersion of a compound I.

44. A pharmaceutical composition of claim 42 additionally comprising a carrier.

45. A pharmaceutical composition of claim 44 wherein said carrier comprises mannitol.

46. A pharmaceutical composition of claim 43 comprising a suspension in oil.

47. A pharmaceutical composition of claim 38 for oral administration.

48. A pharmaceutical composition of claim 47 additionally comprising a solid carrier.

49. A pharmaceutical composition of claim 47 additionally comprising gelatin and a plasticiser.

50. A pharmaceutical composition of claim 38 for rectal administration.

51. A pharmaceutical composition of claim 50 additionally comprising a suppository base.

52. A pharmaceutical composition of any of claims 38 to 51 which further comprises one or more additional active agents, for example another anti-cancer agent, e.g. as defined in claim 37.

53. A process for the preparation of a compound of the formula 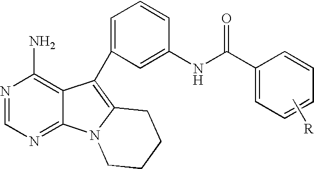
wherein
R=a substituent,
which process comprises the following reaction scheme:

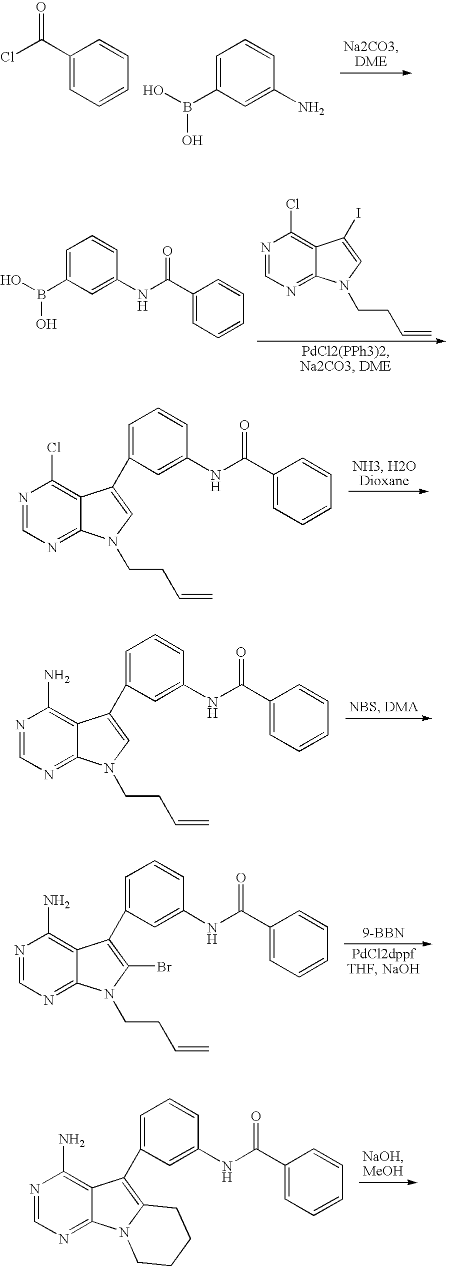

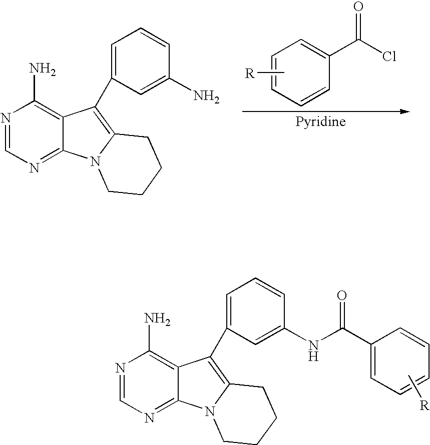

54. A process of claim 53 wherein R=3-(,1,1,2,2-tetrafluoroethoxy

55. A compound of the formula 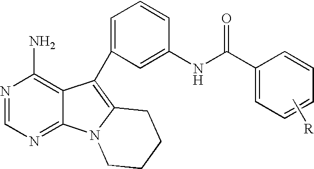
wherein
R=a substituent,
obtained by a process of claim 51.

56. (canceled)

57. (canceled)

58. (canceled)

59. (canceled)

60. A method for treating a tyrosine or serine/threonine kinase or kinase-like-dependent disease in a subject, comprising administering to the subject a therapeutically effective amount of a compound of claim 1.

61. A method for treating in a subject a disease selected from melanoma, angiogenesis, cancer, tumour growth, atherosclerosis, age related macular degeneration, diabetic retinopathy, inflammatory diseases, neurotraumatic diseases, chronic neurodegeneration, pain, migraine or cardiac hypertrophy, comprising administering to the subject a therapeutically effective amount of a compound of claim 1.

62. A method for treating a disease characterized by an activated mutant B-RAF kinase in a subject, comprising administering to the subject a therapeutically effective amount of a compound of claim 1.

63. An intermediate for making compound I of claim 1 of the formula 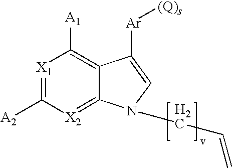
wherein
Q is a group of the formula V¹—W¹ or a moiety comprising an optionally protected functional group capable of being converted to a V¹—W¹ group, as for example in the case of a protected amine capable of being converted, after deprotection, to an amide linker bonded to a V¹ moiety;
v is from 1 to 9, e.g. 2 or 3.
